(19) United States
(12) Patent Application Publication
(10) Pub. No.: US 2023/0190847 A1
(43) Pub. Date: Jun. 22, 2023

(54) PROCESS FOR PREPARING A TOTAL EXTRACT OR A FILTRATE ENABLING THE STABILIZATION OF FRESH PLANT MATTER

(71) Applicant: ID4FEED, Annecy (FR)

(72) Inventors: Camille ROZIER, La Boisse (FR); Francois GAUTIER, Seynod (FR)

(73) Assignee: ID4FEED, Annecy (FR)

(21) Appl. No.: 17/999,267

(22) PCT Filed: May 20, 2021
(86) PCT No.: PCT/EP2021/063412

§ 371 Date: Nov. 18, 2022

(30) Foreign Application Priority Data
May 21, 2020 (FR) FR2005403

(51) Int. Cl. A61K 36/282; A23L 33/105; A23L 33/115; A23K 10/30; A23K 20/158; A61K 47/44; A61K 36/28; A61K 36/8962; A61K 36/54; A61K 36/53; A61K 36/81; A61K 36/288; A61K 36/82; A61K 36/87; A61K 36/9068

(52) U.S. Cl. CPC A61K 36/282; A23L 33/105; A23L 33/115; A23K 10/30; A23K 20/158; A61K 47/44; A61K 36/28; A61K 36/8962; A61K 36/54; A61K 36/53; A61K 36/81; A61K 36/288; A61K 36/82; A61K 36/87; A61K 36/9068; A61K 2236/33; A61K 2236/51; A61K 2236/15; A61K 2236/37

(57) ABSTRACT

A process for preparing a total extract which is a mixture of a fresh plant matter with a fatty matter includes a. contacting the fresh plant matter which comprises at least 10% of water by weight relative to its total weight before or after desiccation loss, with the fatty matter chosen from hydrogenated oil or fat at a temperature between 50° C. and 180° C.; b. grinding the plant matter-fatty matter mixture at said temperature between 50° C. to 180° C.; and c. recovering a total extract that is solid or liquid at ambient temperature with a concentration of less than or equal to 4% of water by weight relative to the total weight of the total extract. Extracts recovered from such process and treatments using the extracts are also disclosed.

### TECHNICAL FIELD

This invention relates to a process for preparing a solid or liquid totum or filtrate at room temperature, from a fresh plant material and a fatty material, as well as the low-water totum or the liquid or solid filtrate that could be obtained according to said process, and their uses.

A totum can be defined as a mixture of at least one plant material with at least one solid or liquid fatty material at room temperature. Thus, the totum comprises active compounds or metabolites, which are in particular extracted from the plant material and can be transferred in part to the fatty material and in part to the plant residue. Active compounds or natural metabolites are molecules originating from a plant material or part of a plant material, whose biological and technological activities have been demonstrated and described in the literature. These natural active compounds can be in pure form or contained in extracts in which residue and solvent are separated. The interest of these active compounds can be established in the context of food and/or welfare and/or human or animal health. Their use as additives can cover a variety of purposes, such as:
improving health (antioxidant, anti-inflammatory, anti-microbial compounds, alkaloids and polyphenols);
improving palatability (compounds that increase palatability such as aromatic compounds, terpenes or pigments such as carotenoids or chlorophylls); and
contribution to nutrition (nutrients such as proteins, amino acids, vitamins, trace elements, etc.).

The natural metabolites of plant origin to which it is possible to attribute biological activities of interest in food and human or animal health may belong to different families of molecules. These are mainly secondary metabolites, which, unlike primary metabolites, are not directly essential for plant nutrition, growth and development (Verpoorte, 2000, Secondary metabolism, In Metabolic engineering of plant secondary metabolism (p.1-29), Springer, Dordrecht). These are compounds whose biosynthetic pathways are fairly specific to a taxonomic group and which generally participate in the interaction mechanisms between the plant and its environment (defence, resistance and responses to abiotic and biotic stresses, symbioses, allelopathy, etc.).

There are different families of secondary metabolites of interest in animal nutrition and health.

The first is alkaloids (compounds that are generally alkaline and contain at least one nitrogen atom). These are compounds that generally have a significant biological activity, in particular an action on the central and/or peripheral nervous system (stimulant or depressant), notably as anaesthetics, as hypertensive agents or anti-hypertensive agents, as anti-malarial drugs or as anti-cancer drugs.

Alkaloids are generally grouped according to their nucleus (non-heterocyclic, indole derivative, pyrrole, pyridine, tropane, etc.). Alkaloids include well-known molecules such as caffeine, morphine, piperine, nicotine, atropine, scopolamine and quinine.

Capsaicinoids, including capsaicin and dihydrocapsaicin, can account for up to 90% of total capsaicinoids. These are the active components of the chilli pepper which belong to the benzylamine group of alkaloids. Consumption of capsaicin activates TRPV1 receptors which activate a burning sensation. It also stimulates the production of two hormones, adrenaline and noradrenaline, and therefore has therapeutic value given its anti-inflammatory, antioxidant and analgesic properties (Zimmer et al., 2012, Antioxidant and anti-inflammatory properties of Capsicum baccatum: from traditional use to scientific approach. Journal of Ethnopharmacology, 139(1), 228-233).

Then there are the carotenoid pigments (yellow, orange or red tetraterpenes), including carotenes, which are composed solely of carbon and hydrogen, and xanthophylls, which also contain oxygen atoms.

Chlorophylls (a, b, c1, c2 and d) are pigments present in all green plants (terrestrial and aquatic). Chlorophyll a (C55H72O5MgN4) is still the most common form found in plant leaves.

Anthocyanins are water-soluble pigments (oxygenated heterosides) that range from red to blue.

Curcuminoids (orange pigments from the rhizome of Curcuma longa), have been shown to significantly decrease concentrations of C-reactive protein, an important factor in inflammation (Sahebkar, Are Curcuminoids Effective C-Reactive Protein-Lowering Agents in Clinical Practice? Evidence from a Meta-Analysis, Phytother Res, 2013 Aug. 7).

Flavonoids can range in colour from red to ultraviolet depending on the pH and consist of two aromatic rings linked by three carbons.

These different classes of pigments have mainly inflammation-regulating and light-protecting effects and also act as antioxidants (powerful anti-free radicals) (Stahl and Sies, Bioactivity and protective effects of natural carotenoids. Biochimica et Biophysica Acta (BBA)-Molecular Basis of Disease, 1740(2), 101-107).

The interest of their use in animal feed is notably to couple their antioxidant activities with their participation in improving the visual quality (colouring and appearance) of the product formulation, as well as in the colouring and preservation of animal products (meat, eggs).

Terpenes are also interesting secondary metabolites. These are volatile compounds with an aromatic ring structure and hydroxyl and terpenoid groups. They are the source of the aromatic properties of certain plants according to their taxonomy. According to the literature, there are about 25,000 different terpene structures.

In addition to this, the properties of another family, the phenols, are essential components of essential oils.

Phenols are the metabolites that give essential oils their very characteristic smell and biological activities.

For example, oregano essential oil is composed mainly of thymol (phenol monoterpenoids) and its isomer, carvacrol and y-terpinene, the presence of which gives the essential oil its antioxidant and antimicrobial properties.

Natural compounds of plant origin have a wide range of applications in the fields of cosmetics and perfumery, but also in health and human and animal nutrition. Prior art

They are generally obtained in a first step by harvesting, drying, storing and packaging the raw plant material.

Document FR2943684 describes, for example, a process for extracting non-volatile natural compounds contained in a solid raw material of natural origin, in particular a plant, in a dispersible form, using a natural fat or a mixture of natural fats, in particular a vegetable oil or a mixture of vegetable oils, characterised in that it comprises:
a) one step of mixing and impregnating the solid raw material in dispersible form with the natural fat at a temperature above the melting point of the fat and in an atmosphere free or essentially free of oxygen,
b) one step of micro-dispersing the solid material and possibly breaking the cells of the raw material, in the natural fat at a temperature above the melting point of the fat, and
c) one step of heating the mixture at high temperature.

Document FR3013979 describes a process for preparing a totum comprising the following steps:
(a) Preparing dehydrated grapes in a form which is dispersible in oil at a temperature above the melting point of the oil;
b) Mixing the solid material obtained in step a) with the oil, or a mixture of oils;
c) Heating and physically processing the mixture by implementing:
at least one step of micro-dispersing the solid material and breaking the cells of the raw material in the oil at a temperature above the melting point of the oil;
at least one step of heating the mixture to a high temperature, advantageously between 80°-200° C., for a very short time, and
d) Recovering the oily composition from step c).

Document EP3290499 describes a process for producing a totum, containing 3% by weight or less of water, comprising:
one step which consists of bringing a fat into contact with dried rosemary leaf powder;
one step of filtering after the rosemary contact step;
one step of deodorising by heating to a temperature of 170° C. is carried out after filtration.

Such documents do not therefore describe an “all-in-one” process for drying, grinding, green extraction, formulation, and stabilisation of a fresh plant material in a solid fatty material at room temperature to obtain a solid totum at room temperature comprising active compounds or metabolites extracted in particular from the plant material and which can be partly transferred into the fatty material and partly into the plant residue.

The drying phase is a critical phase for any plant metabolite (Mediani et al., 2014, Effects of different drying methods and storage time on free radical scavenging activity and total phenolic content of Cosmos caudatus, Antioxidants, 3(2), 358-370). It has been shown that a very significant loss of the active molecules contained in the plant (evaporation, degradation, metabolisation) was observed during this drying step, consequently decreasing the biological activity potential of the plant (e.g. antioxidant, antimicrobial) (Lim and Murtijaya, 2007, Antioxidant properties of Phyllanthus amarus extracts as affected by different drying methods, LWT-Food Science and Technology, 40(9), 1664-1669; Al-Farsi et al, 2005, Comparison of antioxidant activity, anthocyanins, carotenoids, and phenolics of three native fresh and sun-dried date (Phoenix dactylifera L.) varieties grown in Oman, Journal of agricultural and food chemistry, 53(19), 7592-7599).

The degradation of some molecules can also create degradation products that are toxic to cells (O′Brien et al., 2008, Aldehyde sources, metabolism, molecular toxicity mechanisms, and possible effects on human health, Critical reviews in toxicology, 35(7), 609-662).

For plant materials containing a large amount of sugars, the interaction of these with certain amino acids can also be feared during heating (pH-, water-dependent) and produce the synthesis of new molecules modifying the aroma and the biological potential.

Certain drying techniques have been developed to avoid the degradation and loss of molecules that are thermolabile, or that can be hydrolysed or oxidised very quickly. This is the case of low temperature drying (30-38° C.) or freeze-drying which uses a specific state of water, sublimation, in order to dehydrate a product after freezing (Oikawa et al., 2011, Effects of freeze-drying of samples on metabolite levels in metabolome analyses, Journal of separation science, 34(24), 3561-3567; Adams, 1991, Freeze-drying of biological materials, Drying technology, 9(4), 891-925).

However, even using these gentler drying processes, it was shown in a comparison of freeze-dried plant/fresh plant that a significant part of the plant metabolites was lost (Oikawa et al., 2011, Effects of freeze-drying of samples on metabolite levels in metabolome analyses, Journal of separation science, 34(24), 3561-3567). These methods are energy-intensive, time-consuming and economically unviable for companies. However, the main interest in drying plant material lies in the possibility of storing and preserving these dehydrated plants. Removing water from the plant is therefore essential for its conservation, the stabilisation of its chemical content and the preservation of its biological activities over the long term (Mediani et al., 2014, Effects of different drying methods and storage time on free radical scavenging activity and total phenolic content of

Cosmos caudatus, Antioxidants, 3(2), 358-370).

It has also been shown that during storage, and in a manner highly dependent on storage conditions, the chemical profile of a fresh fruit or plant (carotenoids, polyphenols, vitamins, etc.) could be strongly affected and diminished in terms of quantity and quality, as the molecules are not subject to degradation in the same way (Yamauchi and Watada, 1991,

Regulated chlorophyll degradation in spinach leaves during storage, Journal of the American Society for Horticultural Science, 116(1), 58-62; Vishnu Prasanna et al., 2000, Effect of storage temperature on ripening and quality of custard apple (Annona squamosa L.) fruits, The Journal of Horticultural Science and Biotechnology, 75(5), 546-550). Among the extraction processes used, using water as a solvent, are hydrodistillation or steam distillation, cold maceration, hot digestion, decoction, leaching, pressurised or cold percolation, or infusion.

Another common extraction process involves using volatile organic solvents such as petroleum ether, hexane, ethyl ether, acetone, carbon dioxide, benzene or toluene. For the extraction of fresh plant material from a fat, the process traditionally used since ancient times is hot enfleurage. Enfleurage is the process of integrating the aromas of a fresh plant into an oil or fat by maceration. The fatty material can be heated before the process and the plant is infused into it. At the end of the process, the plant is separated from the fatty material by filtration. This process mainly concerns aromatic flowers or herbs.

However, the plant material is not ground during the enfleurage process and the water from the plant material is not removed.

### Technical Problem

In general terms, there is a need to develop a process for preparing a stabilised plant material that allows the fresh plant to be integrated, for example immediately after harvesting, and thus to avoid transport and handling constraints, and to preserve the chemical and biological properties of the plant by avoiding the degradation of active molecules.

In view of the above, one problem that this invention proposes to solve consists in developing a new process for preparing a solid totum or filtrate at room temperature from fresh plant material and fatty material, which is easy and quick to implement and makes it possible to preserve all the metabolic richness of the plant material and to avoid any degradation by oxidising the fragile molecules. The extracted natural active compounds are contained in a complex package and act in synergy with each other, thus increasing the biological potential of the totum. Advantages Provided

The advantage of said process according to the invention is to use the fresh plant material, whole or in part, for example when it leaves the field, directly in the process according to the invention and to grind it, dry it, and stabilise it in the fatty material while preserving the maximum of its biological activities (little metabolic change). Integrating the fresh plant immediately after harvesting is a big advantage for the skilled person in terms of handling, transport but also in terms of preserving the biological properties of the plant material, in particular a plant.

The process makes it possible to stabilise by dehydration, to block the access of oxygen to the active molecules (oxidation) by crushing the plant directly in the fatty material, the latter going to surround the fine plant particles and to bring to the fresh plant material a fatty material with antimicrobial properties in order to avoid the degradation of the active ingredients. In addition to the biological advantage (increasing the biological and chemical potential of the product), stabilisation via this process has an energy advantage as it is an “all-in-one” process. The use of a solid (not liquid) fatty material at room temperature in the process advantageously reduces the penetration of oxygen, light and water and, unlike a liquid oil at room temperature, stabilises the totum even more.

The process can also be used to stabilise co-products from other production processes quickly after they leave the factory. This process is thus also part of the problem of recovery of co/by-products. Indeed, these by-products or co-products are products disposed of by companies which are not or cannot necessarily be stored in optimal conditions, as companies are not compliant for this purpose. The financial burden of disposing of the company's co-products (transport, destruction) is also greatly reduced or eliminated. The idea is therefore to preferably implement the process directly on leaving the factory in order to stabilise the co/by-products as quickly as possible and to keep them in good conditions, so that they can be recovered later. The totum or filtrate from the co/by-products obtained by the process constitutes an optimised stable state in order to store them and recover their chemical richness.

In addition to the advantages in terms of not having to clean different machines, the energy cost is lower than the successive use of several specific tools in that said process is a grinding, dehydrating, mixing, extracting, formulating, all-in-one treatment.

The use of a solid fatty material at room temperature also simplifies the transport of the finished product in that the resulting totum is solid and stable; furthermore, the fact that it is substantially free of water results in a lower weight and therefore a lower transport cost.

Finally, this process is very simple to implement and adaptable (duration of the process, temperature, application or not of a vacuum, grinding, adapted to the initial water content of the plant, to its lignin composition, to its chemical fragility, etc.).

### Technical Solution

The solution to this problem firstly relates to a process for preparing a totum which is a mixture of a plant material with a solid fatty material at room temperature, characterised in that it comprises the following steps according to which:

(a) the plant material is fresh and comprises at least 10% water by mass of water in relation to its total mass (mass/mass) before or after loss to desiccation, alone or as a mixture, in whole or in part, is brought into contact, preferably under stirring, with the fatty material chosen from a fat and a hydrogenated oil, at a temperature of between 50° C. and 180° C.;

(b) the resulting plant material—fat material mixture is then ground at a temperature of between 50° C. and 180° C.;

(c) the ground material obtained from step (b) is heated, preferably under stirring, to a temperature of between 50° C. and 180° C. to dehydrate the mixture; and

(d) a solid totum at room temperature comprising 4% or less water by mass of the total mass of the totum is recovered.

Secondly, it relates to a solid totum or filtrate at room temperature that can be obtained by the process according to the invention.

Thirdly, it relates to the use of a totum or filtrate according to the invention, for the preparation of a food or cosmetic composition.

Fourthly and finally, it relates to a composition comprising a totum or filtrate according to the invention, for its pharmaceutical, nutraceutical or animal health use.

### BRIEF DESCRIPTION OF THE DRAWINGS

The invention and the advantages deriving therefrom will be better understood by reading the following description and the non-limiting methods of implementation, in relation to the annexed figures in which:

FIG. 1 shows the various steps (mixing, grinding, dehydration) of the process according to the invention for the preparation of a totum or filtrate for the stabilisation of fresh plant material.

FIG. 2 shows the various steps (mixing, grinding, dehydration) of a particular embodiment of the process according to the invention for the preparation of a totum or filtrate for the stabilisation of fresh plant material, in which the fresh plant material is input in two steps.

FIG. 3 shows a picture of a totum obtained by the process according to the invention using fresh Habanero chilli and monolaurate glycerol.

FIG. 4 represents the quantification of carotenoids identified in the samples of fresh Habanero chilli, oven dried at 100° C. and dehydrated according to the process according to the invention implemented in Example 5.

FIG. 5 represents more particularly the absolute quantification of different carotenoids of interest in animal nutrition/health (lutein; β-carotene at zeaxanthin) identified in the samples of fresh Habanero chilli, oven dried at 100° C. and dehydrated according to the process according to the invention implemented in Example 5.

FIG. 6 represents the absolute quantification of capsaicin identified in the samples of Habanero chilli and red chilli, respectively fresh, oven dried at 100° C. and dehydrated according to the process according to the invention implemented in Example 5.

### DESCRIPTION OF EMBODIMENTS

The invention relates to a process for preparing a solid totum or filtrate at room temperature from fresh plant material and fatty material.

According to another embodiment, the invention relates to a process for preparing a liquid totum or filtrate at room temperature, from fresh plant material and fatty material.

A solid at room temperature is a state of matter with its own shape and volume, which can be manipulated and moved without changing its shape or volume, as opposed to a liquid state.

A liquid at room temperature means any other deformable state of matter, regardless of its viscosity, including viscous fluids. As the ions, atoms and molecules are only loosely connected, the liquid takes the shape of the container in which it is placed and flows more or less well depending on its viscosity. As an example of a liquid at room temperature, the liquid has a viscosity of 0.1 cP and 100,000 cP.

Fresh plant material means a living organism belonging to the plant kingdom, including aquatic plants, in whole or in part, comprising at least 10% water by mass of water in relation to its total mass (mass/mass) before or after loss to desiccation, preferably at least 69% (mass/mass), more preferably at least 79% (mass/mass).

In contrast, a dry material generally has a water content that is at most around 5% (mass/mass).

The fresh plant material, alone or in a mixture, in whole or in part, used in the process according to the invention is preferably chosen from fruits, whole plants, aerial parts of plants, roots, bulbs, tubers, seeds such as grape or citrus seeds, skins such as pomegranate or citrus peel, pulps, macerates, oil cakes, or any other by/co-products of plant material such as plant residues or pressed fruits.

The fresh plant material, alone or as a mixture, in whole or in part, used in the process according to the invention is more preferably chosen from wormwood, yarrow, garlic, wild garlic, artemisia, artichoke, pink pepper, goji berry, burdock, basil, coffee, chamomile, cinnamon, blackcurrant, lemon, lemongrass, hemp, coriander, turmeric, cypress, eucalyptus, fenugreek, ash, juniper, clove, ginseng, ginger, pomegranate, hibiscus, hops, laurel, lavender, lemon grass, alfalfa, flax, mint, peppermint, mallow, lemon balm, mustard, white mustard, walnut, hazel, orange, oregano, nettle, onion, paprika, pansy, sweet pepper, chilli, pine, dandelion, pepper, rosemary, grape, savory, sage, wild thyme, marigold, tansy, tea, thyme, clover, goldenrod, most preferably sweet peppers and chillies of the genus Capsicum annuum and frutescens, chillies of the genus Capsicum chinense, garlic, ginger, grape, thyme, paprika, even more preferably sweet peppers and chillies of the genus Capsicum annuum, frutescens and chillies of the genus Capsicum chinense such as Habanero, Bhut Jolokia, Carolina Reaper, Trinidad Scorpion, bird's eye/Thai chillies.

Fresh plant material can usually only be kept in its original state for a short time, e.g. a maximum of a few hours, after harvesting or recovery for by/co-products, otherwise it will degrade and lose much and sometimes all of its active metabolites of interest and associated biological activities.

Advantageously, the plant material will be put into the fatty material directly after harvesting or recovery, or at least as quickly as possible, for example 12 hours, and will preferably be used within 3 hours, in order to preserve all the active metabolites of interest.

The fatty material used in the process according to the invention is chosen from a hydrogenated or non-hydrogenated fat or oil, preferably a hydrogenated or partially hydrogenated fat or oil, solid at room temperature, alone or in a mixture.

A hydrogenated or partially hydrogenated fat or oil that is solid at room temperature means a fat or oil with a melting point above 27° C., preferably above 30° C.

According to a preferred embodiment, insofar as obtaining a solid totum at room temperature is desirable, the fatty material used is preferably a hydrogenated or at least partially hydrogenated fat or oil, solid at room temperature, advantageously with a melting point above 27° C., preferably above 30° C. According to another embodiment, insofar as obtaining a liquid totum at room temperature is desirable, the fatty material used is preferably a liquid oil at room temperature, in particular a non-hydrogenated oil with a melting point above 30° C., preferably above 27° C.

Room temperature means a stable but not necessarily controlled temperature, which is usually 20° C. but can be between 15° C. and 27° C. The fatty material preferably used in the process according to the invention is chosen not only for its efficiency and its interest in the process but also for its cost, its texturing properties and its interest in being used as a food component (energy contribution or antioxidant, antimicrobial or anti-inflammatory biological activities for example).

The fatty material, preferably solid at room temperature, is more preferably chosen from glycerol monolaurate, glycerol monocaprate, glycerol monomyristate, glycerol monopalmitate, glycerol monostearate, almond oil, peanut oil, argan oil, avocado oil, calophyllum oil, safflower oil, rapeseed oil, coconut oil, wheat germ oil, jojoba oil, corn oil, hazelnut oil, apricot kernel oil, virgin olive oil, palm oil, grape seed oil, castor oil, sesame oil, soybean oil, or sunflower oil, the aforementioned oils being hydrogenated or at least partially hydrogenated so that they are solid at room temperature, even more preferentially glycerol monolaurate, glycerol monocaprate, hydrogenated palm oil and hydrogenated sunflower oil, even more preferentially glycerol monolaurate.

In a particularly advantageous way, certain fatty materials used in the process according to the invention are effective solvents, i.e. they allow the transfer of molecules from the plant material into the solvent; this is the case in particular for glycerol monolaurate, glycerol monocaprate and olive oil for example, contrary to for example sunflower oil or palm oil which have a weaker solvent power.

Regardless of their solvent power, the intrinsic properties of the fats or oils used, in particular fats and more particularly glycerol monolaurate, allow the process and the product resulting from the process to be optimised. Fats or oils, preferably hydrogenated or partially hydrogenated fats and oils that are solid at room temperature, create an anaerobic or at least micro-anaerobic environment during the process and inhibit the survival and development of strict aerobic microorganisms. This is intensified by the fact that the hydrogenated or partially hydrogenated fats and oils that can be used for the process are advantageously solid at room temperature and therefore impermeable to air and therefore restrict the colonisation of microorganisms within the totum.

The Applicant was able to demonstrate that glycerol monolaurate, lauric acid monoglycerol (or 2,3-dihydroxypropyl decanoate), comprises a particularly advantageous fatty material according to the invention, with multiple interests. It is solid at room temperature and liquid from 60° C. It is safe for human and animal use. It is thermostable and retains its properties and non-toxicity during the process steps.

Due to its emulsifying properties, its use in the said process facilitates the extraction of water from the fresh plant materials of the totum, the mixture being carried out at a temperature preferably equal to or higher than 60° C. and preferably under constant agitation. The use of glycerol monolaurate in the process helps to remove water from the plant cells. The emulsifying properties of glycerol monolaurate also make it possible to protect the molecules contained in the plant cells from degradation (guided by contact with water or oxygen), through optimised contact with the fatty material. More than heating (drying), the emulsifying properties of glycerol monolaurate also optimise grinding by improving the plant/fatty material/knife contact.

Glycerol monolaurate is a natural fatty acid monoester whose composition (more polar than some vegetable oils) more particularly allows the extraction of amphiphilic compounds such as capsaicinoids. Its use does not pose any risk to humans or animals.

In addition, it shows pronounced antibacterial, antifungal and anti-inflammatory activities. It can be used as an antimicrobial agent and inhibits the growth of Candida strains in vitro and in vivo. It also acts against the growth of Gram+ but also Gram− bacteria such as Staphylococcus, Streptococcus, Gardnerella, Haemophilus but also Listeria monocytogenes. It also acts as a bacteriostatic agent, against Bacillus anthracis, i.e. it blocks its growth without killing the cells. In Staphylococcus aureus, glycerol monolaurate blocks the production of certain exoenzymes and virulence factors such as protein A, α-hemolysin, β-lactamase and toxic shock syndrome toxin 1 (TSST-1).

Its action on the inflammatory cycle was also demonstrated as a parallel and significant decrease in the quantity of pro-inflammatory cytokines (IL-8 and TNF-a) was observed. Glycerol monolaurate can also act in synergy with other products, such as aminoglycosides, notably in the destruction of biofilm of antibiotic-resistant strains of Staphylococcus aureus. Indeed, pre-treatment with glycerol monolaurate would improve the response of biofilms to antibiotics.

Glycerol monolaurate, due to its emulsifying, solvent, physicochemical and antibacterial properties, is the fatty material most preferably chosen for the implementation of the process for preparing a totum or filtrate according to the invention.

For reasons of complexity of implementation, glycerol monolaurate has never been disclosed as a substitute for any extraction solvent in an associated process.

Indeed, although its physico-chemical properties make it an extraction solvent of choice, it is mainly for its antimicrobial (WO2016169129, WO9531966) and anti-inflammatory properties that it has been promoted until now. Glycerol monolaurate has also been described as a constituent of a combination of essential oil molecules used as a preservative for cosmetic products (inhibition of Aspergillus niger, Candida albicans, Staphylococcus aureus, and Pseudomonas aeruginosa) (WO2019047004).

It appears that the antimicrobial properties of glycerol monolaurate have been exploited in various fields of application in food decontamination, infection treatment processes (in human health), as food additives or cosmetic preservatives. Its preservative properties have been verified because the stabilisation of the totum obtained by said process also requires its preservation during storage. The use of glycerol monolaurate as the fatty material in the process (as opposed to hydrogenated palm oil) slowed down or even inhibited the proliferation of microorganisms on the product obtained. Although the antimicrobial properties of glycerol monolaurate have been previously described in the literature, its use as a solvent in an all-in-one process of drying, grinding, green extraction, formulation, and stabilisation of fresh plant material containing active natural metabolites of interest has never been described.

Its antimicrobial activity but also its physico-chemical properties enabling the extraction of a wide range of metabolites make it a particularly preferable candidate, notably as a smart solvent, or solvagent, combining a notable efficiency as a solvent and providing the mixture with the antimicrobial and preservative properties described above.

The process according to the invention comprises a first step (a) in which the fresh plant material comprising at least 10% water by mass of water in relation to its total mass (mass/mass) before or after loss to desiccation, alone or as a mixture, in whole or in part, is brought into contact, preferably under stirring, with the fatty material chosen from a fat and a hydrogenated or non-hydrogenated oil, preferably a fat and a hydrogenated or partially hydrogenated oil which are solid at room temperature, at a temperature of between 50° C. and 180° C., preferably between 60° C. and 130° C., more preferably around 100° C.

The step of bringing the fresh plant material into contact with the fatty material is preferably carried out by constant stirring of between 300 and 2500 revolutions per minute (rpm), preferably under constant stirring of around 500 rpm.

For example, a scraper or mixer ensures constant contact of the fresh plant material, in whole or in part, with the fat and also allows water to be removed during the process.

This contact step, preferably under stirring, at a temperature of between 50° C. and 180° C., preferably between 60° C. and 130° C., more preferably around 100° C., thus consists of a mixing/heating step.

This first step of the process melts the fat, as long as it is solid at room temperature, e.g. glycerol monolaurate or hydrogenated sunflower oil, cooks the fresh plant material in contact with the fat and therefore improves the grinding which is the next step.

This mixing/heating step is essential because it allows the water in the fresh plant material to evaporate, at least in part.

During this step, if the fat used is a solid hydrogenated or partially hydrogenated fat or oil at room temperature, it will, due to the temperature, become liquid and surround and protect the fresh plant material which is subjected to a temperature increase.

The duration of this mixing/heating step depends on the fresh plant material used and its water content.

Advantageously, this step must be carried out in a time necessary and sufficient for the fatty material, which may be solid at room temperature, to be liquefied to allow it to be subsequently ground effectively, in contrast to dry grinding with fat flakes. It can therefore only be a few seconds, especially for a solid fat at room temperature.

Advantageously, insofar as the fatty material used has solvent power, preferably glycerol monolaurate alone or in combination, it also makes it possible to extract, at least in part, i.e. to transfer the metabolites of the fresh plant material into the fatty material.

The fresh plant material is preferably brought into contact with the fatty material for a period of between 5 min and 40 min, for example 10 min, 15 min, 20 min, 25 min, 30 min or 35 min, preferably between 5 min and 20 min, more preferably 10 min.

In step (b), the plant material—fatty material mixture obtained in (a) is then ground at a temperature of at least 50° C., preferably between 50° C. and 180° C., more preferably between 60° C. and 130° C., even more preferably around 100° C.

The grinding step of the plant material—fatty material mixture can last a few seconds, and is preferably carried out for a period of more than 2 minutes, preferably between 3 min and 15 min depending on the plant material, more preferably between 3 min and 5 min.

Grinding is advantageously carried out in such a way that the totum obtained in fine forms a smooth paste, and that the particles of ground plant material remain suspended in the fatty material so that, during cooling, the plant material residue does not settle below. The final totum obtained is homogeneous and contains particles that are invisible to the naked eye.

Grinding is advantageously carried out by serrated knives.

Grinding the fresh plant material into the fatty material is carried out in one or more steps, depending on the type of plant material and its richness in cellulose, lignin, presence of seeds, nuts or bark.

The grinding step of the plant material—fatty material mixture is preferably carried out under stirring between 500 rpm and 3500 rpm, more preferably between 1000 rpm and 2500 rpm, depending on the plant material, even more preferably under constant stirring of around 2500 rpm using, for example, to said scraper to ensure that the ground fresh plant material is in constant contact with the fatty material.

The fresh plant material and/or fatty material, advantageously the fresh material, can be supplied at once at the beginning of the process or alternatively at several times, for example at 2 to 4 times during the process, with the mixing/heating (step a), grinding (step b) (and dehydration (step c)) steps being advantageously carried out after each addition of fresh plant material as illustrated in FIG. 2.

As an illustrative example, ⅓ (mass/mass of total input) of fatty material, preferably glycerol monolaurate, and ⅓ (mass/mass of total input) of fresh plant material, preferably chillies, are added at the start of the process. They are brought into contact with each other under stirring at 100° C. for 10 minutes, then the mixture is ground by knives at 2500 rpm, 100° C., for 5 minutes; again ⅓ (mass/mass of total input) of fresh plant material is added, the mixture is heated for 10 minutes at 100° C., and subsequently the mixture is ground for 5 minutes at 2500 rpm. This allows a higher proportion of fresh plant material to be incorporated into the fat.

According to another illustrative example, ⅓ (mass/mass of total input) of fatty material, preferably glycerol monolaurate, and ⅓ (mass/mass of total input) of fresh plant material, preferably chillies, are input at the beginning of the process. They are brought into contact with each other under stirring at 100° C. for 10 minutes, then the mixture is ground by knives at 2500 rpm, 100° C., for 5 minutes; again ⅓ (mass/mass of total input) of another fresh plant material, advantageously an aromatic plant (e.g. thyme) is added, the mixture is heated for 10 minutes at 70° C., a lower temperature in order to avoid altering the volatile secondary metabolites of aromatic plants (active ingredients) and then the mixture is ground for 5 minutes

The fresh plant material and the fatty material are advantageously used in a final mass/mass ratio of fresh plant material to fatty material of 1:1 to 5:1, preferably 1:1 to 2:1, more preferably 1:1 to 1.5:1, the optimum being dependent on the initial water content of the fresh plant material. In step (c), the mash obtained at the end of step (b) is heated, preferably under stirring, to a temperature of at least 50° C., preferably between 50° C. and 180° C., more preferably between 60° C. and 130° C., even more preferably around 100° C., to dehydrate the mixture, by evaporating the water; in the case where the fatty material used has solvent capacities, this step favours the transfer of the molecules from the plant to the fatty material: this is called green extraction.

The duration of this heating step is dependent on the initial water content of the plant material used in the process. The higher the water content, the longer the heating/mixing time (step c) and water evaporation should be.

This additional heating step is preferably carried out under constant stirring. The heating of the mash is preferably carried out under constant stirring with a scraper at 500 rpm for 5 to 40 min, e.g. 10 min, 15 min, 20 min, 25 min, 30 min, 35 min, preferably 5 min to 20 min, more preferably 10 min, at a temperature of 100° C. in order to continue and optimise the green extraction of the plant material metabolites in the fatty material.

This step is particularly advantageous because it makes the active plant metabolites more bioavailable by making them leave the plant cells, protected nevertheless from potential degradation by their immediate contact with the fatty material.

Also, and particularly advantageously, insofar as the fatty material used has solvent power, it allows further extraction of the metabolites of the plant material in the fat or oil. This is particularly the case when glycerol monolaurate is used as a fatty material, alone or in combination, or olive oil.

According to a particularly preferred embodiment of the process according to the invention, the steps of mixing/heating (step a), grinding (step b), and heating/mixing of the mash (step c), are preferably carried out in darkness.

Darkness means the total absence of light, visible or invisible, natural or artificial. Carrying out the process according to the invention in darkness advantageously protects the molecules from degradation due to light (UV) and thus avoids any loss of molecules of interest by oxidation.

In addition, carrying out the heating/mixing and grinding steps of the fresh plant material directly in the fatty material, and in darkness, reduces the risk of oxidation and loss of active compounds.

As an example of a device allowing the implementation of the process according to the invention, the ROB OQBO 8L® or the HYDROGRIND® can be mentioned, equipped with serrated knives with a rotation speed of between 500 and 3000 rpm, an integrated heating system allowing the products to be heated up to 180° C., and also a 900 mbar vacuum system and a cleaning system. The water that is discharged as steam throughout the process is removed through an outlet on top of the machine.

Such a device therefore makes it possible to control the conditions relating to the atmosphere (O₂/CO₂, ventilation), humidity, temperature and agitation of the fresh plant material used in the process according to the invention. These are important conditions to control when working with fresh plants.

The process according to the invention thus makes it possible to dry fresh plant material and to preserve all its metabolic richness in the hot fat at a temperature of between 50° C. and 180° C., in order to avoid any degradation by oxidation of the fragile molecules.

The grinding of the plants is likewise carried out in the hot fatty material, preferably at a temperature of between 50° C. and 180° C., which facilitates grinding and limits contact of the cellular contents of the plant material with oxygen in the air.

A temperature of at least 50° C., preferably between 50° C. and 180° C., more preferably between 60° C. and 130° C., even more preferably around 100° C., is therefore applied throughout the process.

The fatty material/fresh plant mixture is stirred throughout the process by a scraper rotating at a speed of between 300 rpm and 600 rpm, preferably constantly at 500 rpm,

Advantageously, the process according to the invention is carried out for a total duration of between 13 min and 60 min to minimise energy costs, for example 15 min, 18 min, 20 min, 23 min or 25 minutes.

During the process, water is discharged as steam. Water vapour is released throughout the process. Heating/mixing at a temperature preferably between 60° C. and 130° C., more preferably around 100° C., grinding the plant material into the fatty material, then again heating/mixing preferably between 60° C. and 130° C., more preferably around 100° C., and constant stirring during the process will allow the water to be removed from the plant cells, and ultimately obtain a low-water totum or filtrate. The phenomena of hydrolysis of the plant molecules, entrainment in steam and oxidation are limited by the fact that the plant material is introduced and cooked in the fatty material and that the release of the metabolites takes place, during grinding and mixing at temperatures of preferably 100° C., in the liquid fatty material. In addition, the plant cells are completely surrounded by the liquid fatty material during the process.

The water content of a sample of the dehydrated mash obtained after step (c) is measured, for example, by infrared scale.

At the end of step (c), a solid or liquid, preferably solid, totum at room temperature with a water content of less than or equal to 4% by mass of the total mass of the totum, preferably less than 2.5% (mass/mass), more preferably less than or equal to 1% (mass/mass), is finally recovered in a final step (d).

For example, with this process, the plants are dehydrated (or dried) after only 13 minutes of heating and grinding in fat or oil, advantageously at 100° C., and this up to 99%.

According to another embodiment of the invention, the process may incorporate, at the same time or in an additional step, the application of microwaves and/or ultrasound so as to accelerate the rate of evaporation of water from the plant material and the extraction of plant metabolites in the fatty material having solvent power.

According to a preferred embodiment of the process according to the invention, an additional step of filtering the plant material—fatty material mixture thus obtained is carried out at the end of step (b) or (c). This filtration allows for the removal of plant material residues with a particle size greater than 200 μm or preferably 100μm, before being recovered to obtain a solid or liquid filtrate, preferably solid, at room temperature.

Since the fatty material used is solid at room temperature, the additional filtration step is carried out at a temperature of between 50° C. and 180° C., to obtain a filtrate that is solid at room temperature.

For example, the separation of the filtrate from the plant material residue is achieved by hot (e.g. 90° C.) vacuum filtration or by hot (e.g. 90° C.) centrifugation (e.g. 3000g).

To this end, the invention also relates to a process for preparing a solid or liquid, preferably solid, filtrate at room temperature, characterised in that it comprises the following steps according to which:

a fresh plant material comprising at least 10% water by mass of its total mass before or after loss to desiccation, alone or as a mixture, in whole or in part, is brought into contact, preferably under stirring, with a fatty material chosen from a fat or a hydrogenated or non-hydrogenated oil, at a temperature of between 50° C. and 180° C.;

the resulting plant material—fat material mixture is then ground at a temperature of between 50° C. and 180° C.;

the resulting mash is heated, preferably under stirring, at a temperature of between 50° C. and 180° C., preferably between 60° C. and 130° C., more preferably around 100° C., to dehydrate the mixture;

an additional step of mixing the ground material obtained after step (b) at a temperature of between 50° C. and 180° C., preferably between 60° C. and 130° C., more preferably around 100° C. is carried out;

the resulting dehydrated mash is hot or cold filtered depending on the fatty material used; and

a solid or liquid, preferably solid, filtrate at room temperature comprising a water content of less than or equal to 4% by mass of the total mass of the filtrate, preferably less than 2.5% (mass/mass), more preferably less than or equal to 1% (mass/mass) is recovered, the plant material residues having a particle size greater than 200 μm or preferably 100 μm having been eliminated

The water content of the totum or filtrate which is solid or liquid, preferably solid, at room temperature thus recovered is advantageously measured at the end of the process as is the water activity and will be correlated to the product life (microbial contamination-degradation of actives).

If the fatty material has antimicrobial properties, such as for example advantageously glycerol monolaurate, and the water of the fresh plant material is removed in its entirety, the final totum or filtrate will be optimally stabilised for storage and preservation. If the fatty material becomes solid again at room temperature, the totum is an advantageously formulated product as it is.

To this end, it also relates to a totum or filtrate which is solid or liquid, preferably solid, at room temperature that can be obtained by the process according to the invention.

At the end of the process according to the invention, the totum or filtrate thus obtained is a smooth paste stabilised by its low moisture content and/or by its antimicrobial properties, which can be poured immediately into a suitable container. When using oils or fats that are liquid at room temperature, the resulting totum is liquid with substantially fine dried plant particles resulting from grinding suspended in the oil or fat. The liquid totum can be filtered to remove plant material residues with a particle size greater than 200 or preferably 100 μm and the filtrate used as is or in mixture with other compounds.

This totum or filtrate, the dried plant particles of which are fine, preferably less than 100 μm, is preferably preserved in solid form if the fatty material used is a hydrogenated or partially hydrogenated fat or oil with a melting point above room temperature. The solid filtrate or totum at room temperature can then be remelted for incorporation into a mixture or ground for use in a further process of encapsulation or addition of another material, whether plant or not.

The totum produced in said process is defined as the mixture of dried-dehydrated, ground, possibly green extracted, formulated and stabilised plant material in fatty material.

In the case where the fatty material used has no solvent power and no extraction is made, the totum is a mixture of finely ground plant particles, surrounded by the fatty material. The combination of 2 fatty materials such as glycerol monolaurate and sunflower oil, preferably hydrogenated sunflower oil, allows both extraction with glycerol monolaurate and protection with glycerol monolaurate and sunflower oil.

The filtrate obtained by the process according to the invention is free of particles and essentially comprises the metabolites extracted from the starting fresh plant material. The fact that the fatty materials used can be solid at room temperature reinforces the protection of the plant molecules in the totum or filtrate, and thus the conservation of the totum or filtrate over time. Indeed, the surface of the cooled and solid filtrate or totum in contact with air and light is very limited and therefore the proportion of active metabolites that can be directly subjected to degradation by oxygen, temperature, humidity and light is very limited because the fatty material is impermeable to air. The totum or solid filtrate at room temperature is therefore a stabilised state of the fresh plant material used in the process.

The totum or filtrate obtained according to the invention is low in water, with a water content of less than or equal to 4% by mass of the total mass of the filtrate, preferably less than 2.5% (mass/mass), more preferably less than or equal to 1% (mass/mass), the water of the plant material having been eliminated during said process.

The totum, or filtrate in the case where the fatty material has the capacity, even if limited, to extract, produced by the process according to the invention comprises active compounds extracted from the fresh plant material.

Among the active compounds of interest which are targeted, i.e. preferred, during the application of said process, a family of alkaloids should be mentioned: the capsaicinoids (capsaicin, dihydrocapsaicin, nordihydrocapsaicin).

By the process according to the invention, it is also possible to extract carotenoid pigments (capsanthin, lutein, capsorubin, zeaxanthin, β-carotene, β-cryptoxanthin, β-cryptoxanthin, antheraxanthin) and chlorophylls.

The process temperature may be adapted to extract and not to deteriorate aromatic and volatile molecules such as p-cymene, γ-terpinene, α-pinene, 1,8-cineole, cis-sabinene hydrate, linalool, camphor, borneol, terpinen-4-ol, trans-p-mentha-1(7),8-dien-2-ol, verbenone, bornylacetate, a-terpineol, carvone, thymol, carvacrol, piperitenone, eugenol, α-ylangene, carvacrol acetate, methyl-eugenol, caryophyllene, a-humulene, cis-calamenene, α-calacorene, caryophyllene oxide, 14-hydroxy-(Z) caryophyllene, abetatriene, 14-hydroxy-9-epi-(E) caryophyllene but also certain pigments, vitamines, amino acids or any other molecule that is sensitive to heat.

Indeed, volatile or thermolabile molecules from plants can volatilise or be degraded even at room temperatures (Ormeno et al., Extracting and trapping biogenic volatile organic compounds stored in plant species. TrAC Trends in Analytical Chemistry, 30(7), 978-989, 2011; Flores et al., Nanostructured systems containing an essential oil: protection against volatilization. Quimica Nova, 34(6), 968-972, 2011; Schweiggert et al., Effects of processing and storage on the stability of free and esterified carotenoids of red peppers (Capsicum annuum L.) and hot chilli peppers (Capsicum frutescens L.). European Food Research and Technology, 225(2), 261-270, 2007; Radiinz et al., Study of essential oil from guaco leaves submitted to different drying air temperature. Engenharia na Agricultura, 18(3), 241-247, 2010). Nevertheless, in the case of said process, taking into account the protection by the fatty material, if the conservation in the totum of volatile or thermolabile plant molecules is to be prioritised, the temperature of the process will have to be adapted and not exceed 80° C. Advantageously, the temperature should be between 50° C. and 80° C., more advantageously between 50° C. and 60° C., preferably with the application of a vacuum.

In a particular embodiment of the invention, where thermolabile compounds are of particular interest, a lower temperature can be applied throughout the process (60° C. instead of 100° C.) with the application of a vacuum. The melting and evaporation temperature of the water is reduced by energising the mixture. Water boils at 100° C. under an atmospheric pressure of 1013.25 hPa. At a pressure of 700 hPa, water boils between 90° C. and 91° C. At a pressure of 300 hPa, water boils between 73° C. and 75° C. Thus, reducing the pressure makes it possible, in certain cases of treatment of fragile molecules that do not tolerate high temperatures, to apply a lower temperature and still eliminate the water by evaporation. On the other hand, higher temperatures are sometimes necessary for the green extraction of molecules that are more difficult to extract (due to matrix effects or other reasons) and these higher temperatures also increase the yield of extraction, grinding, water removal and make the fatty material less viscous and therefore easier to handle.

The process is adaptable in terms of temperature, duration of contact between plant material and fatty material, and volume. This makes it possible to extract a range of plant active compounds from a wide variety of plant species. Heat-sensitive compounds such as terpene-type aromatic compounds can thus be extracted and incorporated into the filtrate or totum.

Moreover, all the extracted compounds can act in synergy with each other and with the solvent (intelligent solvent , i.e. with solvent properties that can carry out the green extraction of the active ingredients but also possessing biological properties of interest (antimicrobial) and as a formulation matrix), which is preferably glycerol monolaurate, depending on their properties (antioxidant, antimicrobial, anti-inflammatory, etc.).

Given the physico-chemical properties of glycerol monolaurate, a wide range of molecules (polar, apolar, amphiphilic) can theoretically be extracted into the solvent and contribute to the biological activity of the totum or filtrate (increase in the bioavailability of these molecules extracted from the plant matrix). Examples of such compounds include, but are not limited to, alkaloids, carotenoids, polyphenols, fatty acids, vitamins, bones and amino acids.

Due to the presence of glycerol monolaurate, but also of various pigments extracted during the process, the preferentially obtained totum or filtrate has a smooth, coloured and shiny appearance.

If the extracted plant material comes from an aromatic plant (rosemary or oregano, to name but two), the totum also has the characteristic smell of these plants. The invention also relates to the use of a totum or filtrate according to the invention, for the preparation of a food, preferably for animal nutrition, or cosmetic composition.

Thus, the totum or filtrate charged with compounds of interest can then be formulated according to the needs and target animals and offered for example for their different properties as feed additives for livestock. By way of non-limiting examples, the filtrate or totum is preferably formulated in the form of a powder, granule, pebble, ointment, paste, capsule, microcapsule or tablet.

The invention finally relates to a composition comprising a totum or filtrate according to the invention, for its pharmaceutical, nutraceutical or animal health use.

The preservation during storage and the shelf life of the product is optimised by the fact that it can contain glycerol monolaurate, which is an antimicrobial agent and solid at room temperature, thus protecting the active molecules from oxidation and light at its core.

A nutraceutical product means a totum or filtrate obtained by the process from foodstuffs and formulated, for example, as a powder, granule, pebble, ointment, paste, capsule, microcapsule or tablet, which has a beneficial physiological effect or provides protection against chronic diseases.

### EXAMPLES

The present invention will now be illustrated with the following examples.

### Example 1

### Process According to the Invention of Preparing a Totum or Filtrate

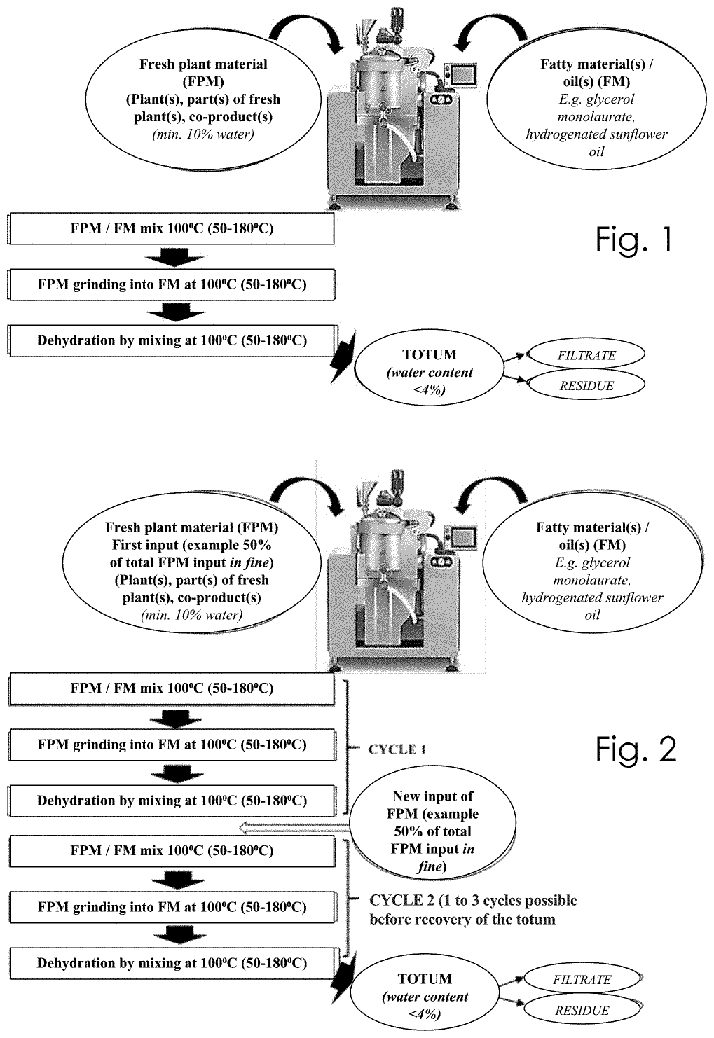

FIG. 1 illustrates the sequence of the various steps inherent in the process of the invention and the resulting products.

Either a first step of bringing into contact fresh plant raw material(s) which may be whole plants, parts of plants (fruits, leaves, etc.), industrial by-products, fresh (with a water content of more than 10%), preferably fruits of the genus Capsicum, and fatty material(s), preferably glycerol monolaurate, and of mixing them at a temperature of 100° C. advantageously (or between 50 and 180° C.).

This is followed by a step of grinding the plant material in the fatty material, again at high temperature (100° C.).

Then a step of mixing and dehydrating the mash by evaporation of the water contained in the latter (ideally at 100° C. or more). The longer the mixing time and therefore the dehydration of the totum, the higher the water content of the fresh plant. The duration of the process should be adapted taking into account the water content of the fresh plant material(s) to be dehydrated and stabilised by the process.

As a result of these steps a totum is obtained with a water content of less than or equal to 4% by mass of the total mass of the totum. The water content of the totum is measured with an infrared scale.

### Example 2

### Embodiment of the Process According to the Invention for the Preparation of a totum or filtrate in which the fresh plant material is input in two steps

FIG. 2 shows an example of a two-step input of fresh plant material. Each input is followed by mixing, grinding and dehydration, i.e. a cycle. This specific case is applied when it is desired to load the totum significantly with plant material. However, it is preferable that the plant supply is processed in at least two steps, as the proportion of plant, before evaporation of the water, must be close to that of the fatty material so that there is contact from the start of the process and the fatty material can surround all the plant cells during the initial mixing.

During the second input, the water in the fresh plant material added at the beginning of the process will have been removed, at least in part, and the volume of plant material thus reduced to make room for a new addition.

The application of vacuum or the increase of the process time can be carried out in order to accelerate the evaporation of the water during said process to obtain a totum with a water content of 4% or less by mass of the total mass of the totum.

### Example 3

### Dehydration of Various Fresh Plant Materials by said Process and Comparison with the Dry Plant Powder Process

Fresh plant materials with a water content of 69% to 94% (Table 1) followed the process of drying/grinding in 2 separate fatty materials at plant material proportions ranging from 70 to 50% (Table 2). The process was carried out in a ROBOQBO® mixer/grinder, equipped with serrated knives at a speed of 500 to 3000 rpm, an integrated heating system allowing the products to be heated up to 120° C. and a 900 mbar vacuum system.

The plant materials and the fats/oils were respectively brought into contact in the defined proportions (Table 2) and heated at a temperature of 100° C. for 10 minutes under stirring between 500 rpm and 3000 rpm.

Grinding was then carried out for 5 or 10 minutes (depending on the plants) still at 100° C.

After grinding, the plant material/molten fatty material mash was stirred with a mixer for 20 or 25 min at 2500 rpm, thus making it possible to prolong the heating of the mash, the evaporation of water, and in the case where the fatty material has solvent properties, the transfer of the plant metabolites to the fatty material.

The water vapour was evacuated throughout the process through an air outlet on the top of the machine.

At the end of the process, the resulting smooth paste is immediately poured into aluminium trays before solidifying at room temperature. Moisture measurement was carried out with an infrared scale (and validated by oven drying the plant materials and products).

The process was also carried out with a dry chilli powder with a water content of 5.11% and glycerol monolaurate (50:50) as a control.

TABLE 1
Measurement of the water content of the plant material used

| Fresh fruit | Water content |
|---|---|
| Red chilli | 87% |
| Green chilli | 92% |
| Habanero chilli | 91% |
| Garlic | 69% |
| Ginger | 94% |
| Grape | 80% |
| Thyme | 79% |

TABLE 2

| Product obtained after 40 min at 100° C. | Totum water content |
|---|---|
| Fresh R. chilli + GML 50:50 | 2.40% |
| Fresh G. chilli + GML 50:50 | 1% |
| Fresh Habanero (G. and R.) + GML 50:50 | 2.12% |
| Fresh G. chilli + hydrogenated palm oil 50:50 | 0.63% |
| Fresh R. chilli + hydrogenated palm oil 50:50 | 0.79% |
| Fresh G. chilli + GML 70:30 | 0.71% |
| Fresh ginger + GML 50:50 | 0.77% |
| Fresh garlic + GML 50:50 | 0.78% |
| Fresh thyme + GML 50:50 | 0.60% |
| Dry chilli powder + GML 50:50 | 0.23% |

The product from the dry plant powder process has a water content of 0.23% (Table 2).

As illustrated by the results in Table 2, the products obtained by the process according to the invention from fresh plant material and glycerol monolaurate have water contents of 0.6% to 2.4%. The products obtained by the process according to the invention from fresh plants and hydrogenated palm oil have water contents of 0.63% to 0.79%.

Thus, the totum obtained from fresh or dry material is stable because it has a water content of less than 4%. The process according to the invention thus makes it possible to replace the prior drying and grinding of the plant material, a step that is costly in terms of energy and chemical loss, with an all-in-one grinding and dehydration process that ultimately produces a stabilised totum.

### Example 4

### Example of Process Parameters According to the Invention using Fresh Habanero Chilli (91% Water Content) and Glycerol Monolaurate (GML)

The various steps of the process according to the invention are implemented according to the conditions defined in Table 3 below.

TABLE 3
TRIAL PROTOCOL

| ROBOQBO Grinder-Cooker | PROTOCOL | |
|---|---|---|
| Formula: | | |
| GML | 50% = 1000 g | |
| Fresh plant material | 50% = 1000 g | |
| Full Habanero chilli | | |
| Device settings: | | |
| Requested product temperature | 100° C. obtained 98° C. | |
| Grinding mixer speed | 3000 | rpm |
| Grinding speed | 5 | min |
| Cooking Speed | 2500 | rpm |
| Cooking time before grinding | 10 | min |
| Cooking and mixing time after grinding | 20 | min |
| Temperature of raw materials on entry | 19° | C. |

| | |
|---|---|
| Use of 700 mbar vacuum | NO |
| Product characteristic: | |
| Product appearance | Smooth with no seed particles |
| Organoleptic aspect: spicy sensation, | Ambiance 2, in the mouth 6 |
| scale from 1 to 10 | |
| Humidity moisture meter 1 | 2.12% |

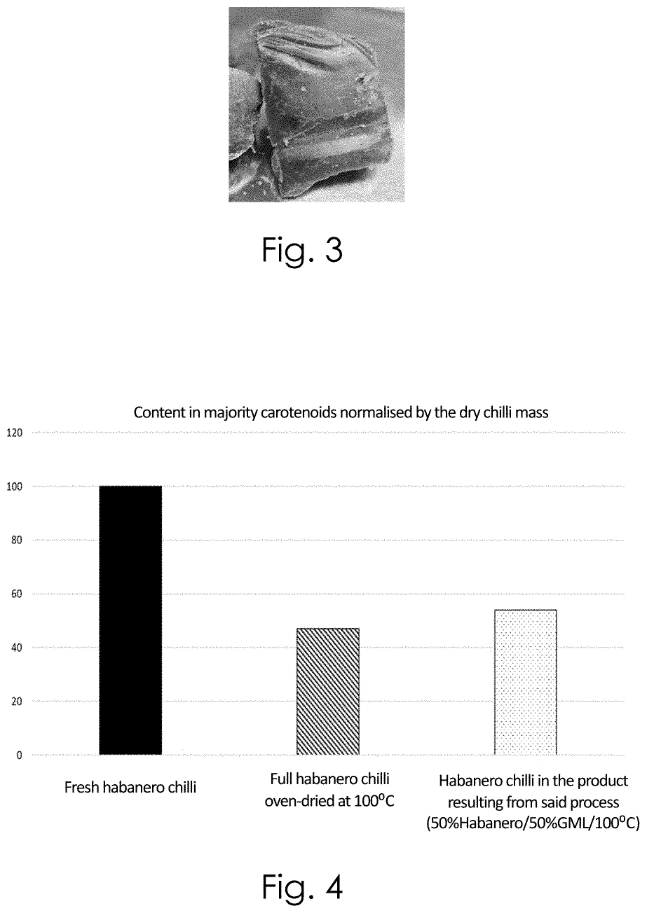

The resulting totum has a water content of around 2% and has the appearance of a smooth, homogeneous paste as shown in FIG. 3 with a strong spicy sensation.

### Example 5

### Measurement of the Impact of the Process on the Metabolites of Interest Extracted from Chillies

The process described below was carried out using Habanero chillies, the water content of which is shown in Table 1 above. The results of chemical analysis of the carotenoids extracted from the resulting totum are highlighted below. Process implemented:

Glycerol monolaurate (GML) (1 kg) and whole Habanero chilli fruits (1 kg) were introduced into the ROBOQBO® and heated (cooked) at 100° C. for 10 min.

This was followed by a grinding step lasting 5 minutes at 100° C. (knives throughout the grinding step at 3000 rpm).

Following the grinding step, heating (cooking) was prolonged for 20 min at 100° C. with mixing by scrapers at 2500 rpm in order to allow evaporation of the remaining water and, similarly, transfer of the chilli metabolites to the GML (solvent role).

The process therefore has a total duration of 35 minutes.

The resulting totum was hot-cast in a mould, observed in cross-section and then ground to observe its physical behaviour on grinding (viscosity, particle size, colour, odour and pungency).

Due to its ability to be a solvent, glycerol monolaurate allows for a much more colourful totum because the metabolites of the fresh chilli, in this case more specifically the carotenoids, have been green extracted and transferred from the fresh plant material to the fatty material (GML).

The totum with glycerol monolaurate is therefore more attractive to humans and animals The carotenoid dosage (comparison of fresh fruit, fruit dried at 100° C., and product resulting from this process) was thus carried out.

Methanolic extractions were carried out from:

1) fresh Habanero chilli fruit;

2) Habanero chilli whole fruit oven-dried at 100° C.; and

3) the totum from the process, from Habanero chilli, as described above.

Following these extractions, the extracts were analysed by UHPLC-DAD-MS/MS. Carotenoids were identified by their representative DAD spectrum. Some carotenoids could be quantified by parallel analysis of a standard range of the analytical standard. Each assay was standardised to be equivalent to the dry mass of the chilli and to allow comparison of the potential loss of metabolites following drying.

The comparative quantification of the carotenoids identified in the samples of fresh Habanero chillies, oven-dried at 100° C. and dehydrated according to the process according to the invention, is illustrated in FIG. 4 representing the sum of the areas of the following metabolites (UHPLC-MSMS, MRM identification mode): β-carotene; β-cryptoxanthin; zeaxanthin; U_3.44; capsanthin; capsorubin; U_5.75; U_10.51; putative capsanthin myristate;

putative capsanthin palmitate; putative capsorubin laurate myristate; putative violaxanthin dimyristate; U_11.19; putative capsanthin myristate palmitate; lutein.

According to the results thus obtained, the sum of identified carotenoids is much

higher, as expected, in the fresh fruit (Habanero chilli). On the other hand, advantageously and not obviously, the sum of the identified carotenoids is in greater quantity in the totum resulting from the process according to the invention (54% of the fresh fruit) than in the chilli dried in the oven at 100° C. (47% of the fresh fruit).

Thus, dehydration by the process according to the invention protects this class of molecules overall from degradation by heat or at least does not cause more degradation than during oven dehydration, for a lower energy cost provided by the all-in-one process.

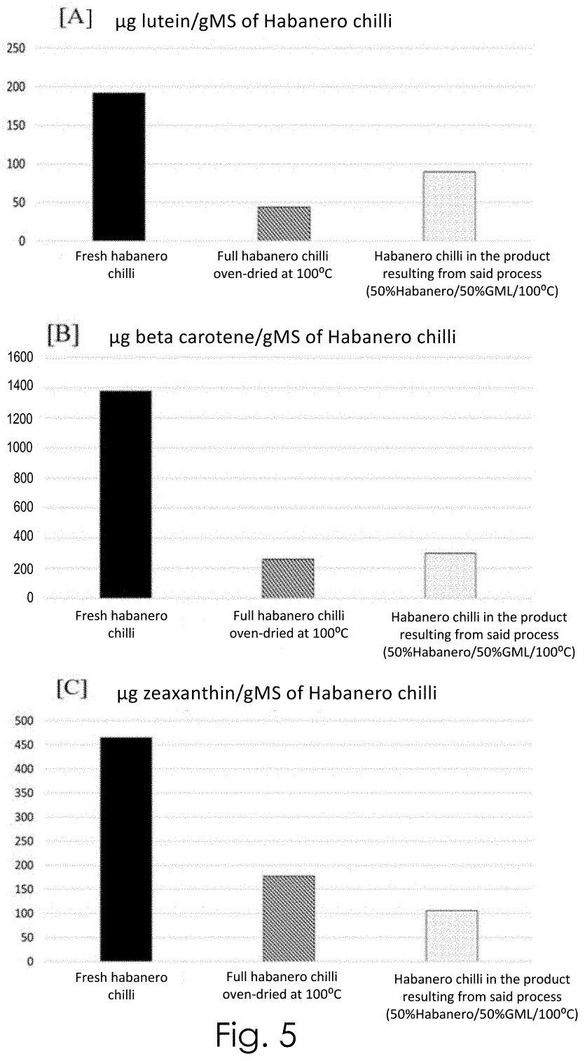

More particularly, the absolute quantification (μg/g dry chilli material) different carotenoids of interest in animal nutrition/health, classed by order of interest (A) lutein; B) carotene; C) zeaxanthin) identified in the samples of fresh Habanero chillies, oven dried at 100° C. and dehydrated according to the process according to the invention, is illustrated in FIG. 5.

According to the results thus obtained, lutein and β-carotene, like most of the carotenoids studied in green, red and Habanero chilli products are in higher concentration in the fresh fruit. On the other hand, advantageously and not obviously, the quantity of these carotenoids of interest is greater in the totum resulting from the process according to the invention than in the oven-dried chilli.

In a more isolated way and without any real explanation except perhaps bonds with the fatty material (matrix effect due to their polarity, bonds with glycerol monolaurate and trapping by the matrix) which could prevent their total extraction, some metabolites (zeaxanthin) are present in greater quantity (in the fresh fruit and) the dried fruit in comparison with the totum resulting from the process.

The dosage of capsaicin, the main alkaloid giving chilli pepper its pungency and of interest in animal nutrition/health for, in particular, its anti-inflammatory potential, was also carried out (comparison of fresh fruit, fruit dried at 100° C., and product resulting from said process).

Methanolic extractions were carried out from:

1) fresh Habanero chilli fruit on the one hand and red chilli on the other;

2) whole fruit oven-dried at 100° C. of Habanero chilli on the one hand and red chilli on the other hand; and

3) the totum resulting from the process, from Habanero chilli on the one hand and red chilli on the other hand, as described above.

The extracts were also analysed by UHPLC-DAD-MS/MS as described in the carotenoid analysis section. Each assay was standardised to be equivalent to the dry mass of the chilli and to allow comparison of the potential loss of metabolites following drying.

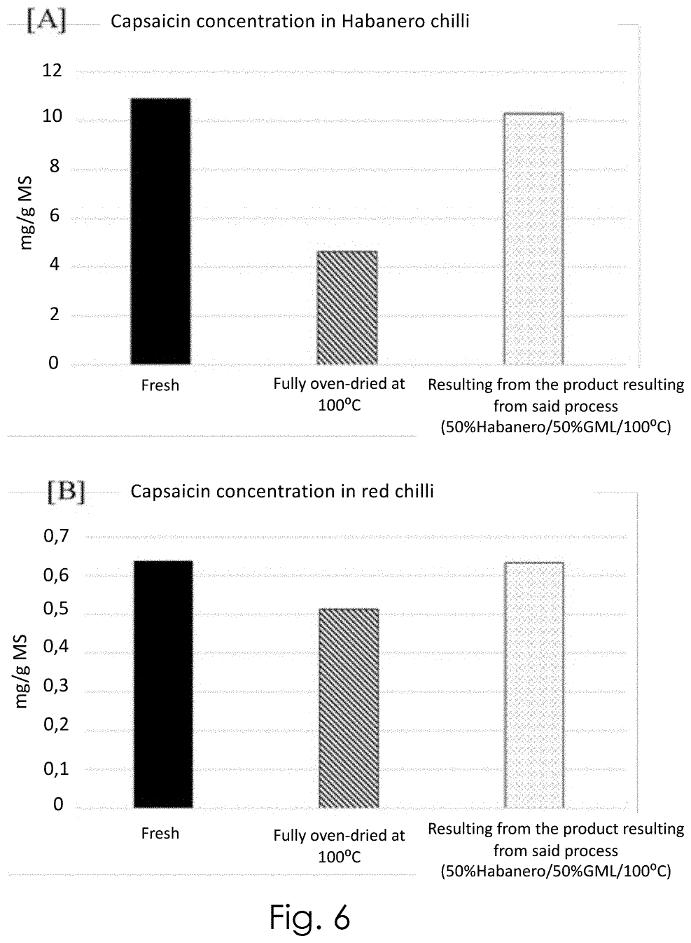

The absolute quantification of capsaicin in samples of fresh Habanero and red chillies, oven dried at 100° C. and dehydrated according to the process according to the invention, is illustrated in FIG. 6 (A-case of Habanero chillies and B-case of red chillies) representing the amount of capsaicin in mg/g (UHPLC-MSMS, MRM identification mode).

Habanero chilli contains a high level of capsaicin (10.9 mg/g DM) compared to red chilli (0.67 mg/g DM).

In the case of both Habanero and red chillies, the same trend was observed. The capsaicin concentration of oven-dried chilli at 100° C. is significantly lower than that of fresh chilli (43% and 81%, in fresh chilli dry mass equivalent, for Habanero and red chilli respectively).

Interestingly and very advantageously, the capsaicin content of the totum resulting from said process (in dry mass equivalent of chilli pepper contained in the totum) is almost equivalent to the content of fresh chilli pepper, i.e. 95% for Habanero chilli pepper and 99% for red chilli pepper.

In summary, according to the results thus obtained, the capsaicin studied in Habanero and red chillies is in higher concentration in the fresh fruit but almost equally so in the totum resulting from the process. A very low loss is therefore observed during the dehydration of the chilli fruits by said process in total opposition to the oven drying of the fruits which induces a loss of between 19% and 57% of the capsaicin initially contained in the fresh fruit depending on the genetics of the chilli treated.

### Example 6

### Microbiological Study on the Preservation of Totum

The process described in Example 1 has been implemented.

The results of the microbial growth observed from suspensions of totum obtained from:

1) 50% palm oil/50% fresh Habanero chilli; and

2) 50% glycerol monolaurate (GML)/50% fresh Habanero chilli; are compiled in Table 4 below.

TABLE 4

| | T + 2 days | T + 15 days |
|---|---|---|
| 50%/GML/50% | 5 bacterial colonies | No bacterial or |
| fresh habanero | (UFC) | fungal colony |
| chilli product | Fungal colony not | |
| | observed | |
| 50% palm oil/50% | 66 bacterial colonies | 340 bacterial colonies |
| fresh habanero | (UFC) | Around 80 fungal colonies |
| chilli product | 7 fungal colonies | (difficult to count due |
| | | to the spread of colonies) |

Thus, due to its solvent properties (extraction of plant metabolites and antimicrobial properties), glycerol monolaurate is a preferred fatty material for fresh plant stabilisation and totum preservation (storage). The totum obtained with palm oil may require the use of preservative(s).

### Example 7

### Process Implemented using fresh Habanero Chilli in Glycerol Monolaurate with the Addition of Plant Material in Two Steps

The various steps of the process (with the input of plant material in two steps, i.e. the realisation of two cycles as illustrated in FIG. 2) according to the invention are implemented according to the conditions more particularly defined in Table 5 below.

TABLE 5
TRIAL PROTOCOL

| ROBOQBO Grinder-Cooker | PROTOCOL | |
|---|---|---|
| Formula: | | |
| GML | 500 | g |
| Fresh plant material | 1000 g (500 g cycle | |
| Full Habanero chilli | 1 + 500 g cycle 2) | |
| Device settings: | | |
| Product temperature | 110° | C. |
| Cooking time before grinding | 10 | min |
| cycle 1 | | |
| Cooking speed cycle 1 | 500 | rpm |
| Grinding time cycle 1 | 10 | min |
| Grinding mixer speed cycle 1 | 3000 | rpm |
| Cooking and mixing time after | 10 | min |
| grinding cycle 1 | | |
| Cooking time before grinding | 10 | min |
| cycle 2 | | |
| Cooking speed cycle 2 | 500 | rpm |
| Grinding time cycle 2 | 10 | min |
| Grinding mixer speed cycle 2 | 3000 | rpm |
| Cooking and mixing time after | 10 | min |
| grinding cycle 2 | | |
| Temperature of raw materials | 17° | C. |
| on entry | | |

| | |
|---|---|
| Use of 700 mbar vacuum | NO |
| Product characteristic: | |
| Product appearance | Dark red colour - colour and odour |
| | more pronounced than for the 50% |
| | GML/50% fresh habanero product |
| Humidity moisture meter 1 | 4.00% |

The totum thus obtained has a water content of around 2% and has the appearance of a smooth, homogeneous paste, dark red in colour, with a more pronounced colour and odour than the totum obtained according to example 4 (with the addition of the chilli at one time and the completion of a single cycle).

1. A process for preparing a totum which is a mixture of a plant material with a solid fatty material at room temperature, the process comprising:
(a) contacting a plant material that is fresh and comprises at least 10% water by mass of water in relation to its total mass (mass/mass) before or after desiccation loss, alone or as a mixture, with a fatty material chosen from a fat and a hydrogenated oil, at a temperature of between 50° C. and 180° C. to produce a plant material-fat material mixture;
(b) grinding the plant material-fat material mixture at a temperature of between 50° C. and 180° C. to form a ground material;
(c) heating the ground material to a temperature of between 50° C. and 180° C. to dehydrate the mixture to form a dehydrated mash; and
(d) recovering a solid totum at room temperature comprising 4% or less water by mass of the total mass of the totum.

2. The process according to claim 1, comprising filtering the dehydrated mash to recover a solid filtrate at room temperature.

3. The process according to claim 1, characterised in that wherein (a), (b) and (c) are performed in the dark.

4. The process according to claim 1 wherein the fresh plant material is selected from the group consisting of fruits, whole plants, aerial parts of plants, roots, bulbs, tubers, seeds, skins, pulp, macerates, cakes, or any other a plant material by-product, a plant co-product, and mixtures thereof.

5. The process according to claim 4, wherein the fresh plant material is selected from the group consisting of wormwood, yarrow, garlic, wild garlic, artemisia, artichoke, pink pepper, goji berry, burdock, basil, coffee, chamomile, cinnamon, blackcurrant, lemon, lemongrass, hemp, coriander, turmeric, cypress, eucalyptus, fenugreek, ash, juniper, clove, ginseng, ginger, pomegranate, hibiscus, hops, laurel, lavender, lemon grass, alfalfa, flax, mint, peppermint, mallow, lemon balm, mustard, white mustard, walnut, hazel, orange, oregano, nettle, onion, paprika, pansy, sweet pepper, chilli, pine, dandelion, pepper, rosemary, grape, savory, sage, wild thyme, marigold, tansy, tea, thyme, clover, goldenrod, and combinations thereof.

6. The process according to claim 5, wherein the fresh plant material is selected from the group consisting of sweet peppers and chillies of the genus Capsicum annuum, frutescens, chillies of the genus Capsicum chinense, garlic, ginger, grape, thyme, paprika, and combinations thereof.

7. The process according to claim 6, wherein the fresh plant material is selected from the group consisting of chillies of the genus Capsicum annuum, frutescens, and chillies of the genus Capsicum chinense, and combinations thereof.

8. The process according to claim 1, wherein the fat or hydrogenated oil is selected from the group consisting of glycerol monolaurate, glycerol monocaprate, glycerol monomyristate, glycerol monopalmitate, glycerol monostearate, almond oil, peanut oil, argan oil, avocado oil, calophyllum oil, safflower oil, rapeseed oil, coconut oil, wheat germ oil, jojoba oil, corn oil, hazelnut oil, apricot kernel oil, virgin olive oil, palm oil, grape seed oil, castor oil, sesame oil, soybean oil, sunflower oil, and combinations thereof.

9. The process according to claim 8, wherein the fat or hydrogenated oil is selected from the group consisting of glycerol monolaurate, glycerol monocaprate, hydrogenated palm oil, hydrogenated sunflower oil, and combinations thereof.

10. The process according to claim 9, wherein the fat or hydrogenated oil is glycerol monolaurate.

11. A solid totum or filtrate at room temperature obtained by the process according to claim 1.

12. (canceled)

13. A composition comprising a totum or filtrate according to claim 11, wherein the composition is in the form of a pharmaceutical, a nutraceutical or an animal health composition.

14. The process according to claim 1, wherein contacting the plant material with the fatty material comprises stirring.

15. The process according to claim 1, wherein heating the ground mixture includes stirring while heating.
